(19) United States
(12) Patent Application Publication
(10) Pub. No.: US 2008/0227170 A1
(43) Pub. Date: Sep. 18, 2008

(54) Use of Pit Emulsions in Biocatalytic Reactions

(76) Inventors: Albrecht Weiss, Langenfeld (DE); Eric Dubreucq, Montpellier (FR); Jean-Pierre Molitor, Buthiers (FR)

Correspondence Address: COGNIS CORPORATION;PATENT DEPARTMENT, 300 BROOKSIDE AVENUE, AMBLER, PA, 19002, US

(21) Appl. No.: 11/816,419

(22) PCT Filed: Feb. 7, 2006
(86) PCT No.: PCT/EP2006/001041

§ 371 Date: May 6, 2008

(30) Foreign Application Priority Data
Feb. 16, 2005 (DE) 102005007174.0

(51) Int. Cl. C12N 9/02

(52) U.S. Cl. 435/189; 516/53

(57) ABSTRACT

Oil-in-water emulsions produced by the phase inversion temperature process, with droplet sizes of 50 to 400 nm, for use as the reaction medium for biocatalytic reactions.

### FIELD OF THE INVENTION

This invention relates to the use of emulsions produced by the PIT (phase inversion temperature) process as a reaction medium for biocatalytic reactions.

### BACKGROUND AND RELATED ART

Enzymes are being increasingly used as catalysts in chemical and biochemical syntheses with hydrolases and especially lipases (EC 3.1.1.3) already being used in many industrial fat-splitting and transesterification processes by virtue of the often milder reaction conditions their use permits. Suitable biocatalytic synthesis processes are described, for example, in K. Drauz and H. Waldmann, Enzyme Catalysis in Organic Synthesis, WILEY-VCH, Vol. I to III, 2002; and U. T. Bornscheuer and R. J. Kazlauskas in Hydrolases in Organic Synthesis with industrial transformations and applications thereof being described by A. Liese, K. Seelbach and C. Wandrey in Industrial Biotransformations, WILEY-VCH, 2002.

The significance of using enzymes in organic solvents, particularly for the production of fine chemicals, continues to grow. In the two-phase or multi-phase systems used, water-soluble and water-insoluble reaction components can be usefully integrated by this method into an overall system to facilitate an improved mass transfer. Often, a reaction component is actually made available for an enzymatic attack by such systems, particularly when one or more starting products is/are solid at the reaction temperature.

In the multi-phase reaction systems used for these biotransformations, both the isolated enzymes and whole microorganisms are frequently used. Enzymes and microorganisms in the form of the whole cells or in the form of active cell constituents are referred to hereinafter as “biocatalysts”.

A common disadvantage is that the solvents used adversely affect the reactivities of the catalysts used (whether enzymes, whole microorganisma or cell constituents thereof), leading to the denaturing of the biocatalysts, diminishing or totally destroying their performance. The efficient reaction of the components by the biocatalysts, which are generally present in aqueous phase, requires a large interface (formed when small droplets form a phase, for example, by agitation or homogenization) between the hydrophilic and the hydrophobic phases.

Mixing/stirring is necessary for accelerating these biocatalytic reactions, with some reactions, such as oxidation reactions, for example, also requiring gassing. An unwanted effect of gassing is foaming which can cause problems with these reactions as biocatalysts may be inactivated at the interfaces of the foams.

On the one hand, intensive mixing is required for an efficient reaction; on the other hand, it can be harmful. Accordingly, it is essential to have a system which is capable of providing very large interfaces for a low shear rate, a low stirrer speed or moderate mixing conditions.

The problem with biocatalytic reactions often lies in the availability and stability of the catalysts involved in the process. There are known enzymes or microorganisms which are stabilized by immobilization, for example by microencapsulation, and which can be used several times. However, the demand continues for new and better biocatalysts for commercial applications, including frequent demands for new biocatalysts with suitable stability. More modern methods make use, for example, of “directed evolution” to develop the desired profile of the biocatalysts.

The reaction of hydrophobic compounds may be carried out simply by using water-in-oil (w/o) microemulsions as described by Orlich and Schomaeker in Enzyme Microb. Technol.; 2001; 28; 1; 42-48 for the lipase from Candida rugosa. However, the concentration of water in the solution and the composition of the components of the w/o microemulsion are very critical to a successful reaction.

Accordingly, the problem addressed by the present invention was to provide a system for biocatalyzed reactions in which solvents that can damage the biocatalyst would be avoided and substances poorly soluble in aqueous systems could still be reacted. In addition, the reaction would take place under mild mixing conditions in order to limit or avoid the negative effects described above.

The substrate concentrations in the system would be capable of variation although the large interface and hence the concentrations of oil and water would remain constant to the extent they would have no major influence on the reaction or on the activity of the enzyme. In addition, the systems would be inexpensive and recyclable and would have little or no adverse effect on the stability of the biocatalyst. Accordingly, it would even be possible to use enzymes which show too little stability in conventional organic solvent systems.

### BRIEF SUMMARY OF THE INVENTION

The present invention relates to the use of o/w (oil-in-water) emulsions as a reaction medium for biocatalytic reactions containing at least water, one or more emulsifiers and an oil phase, the emulsion being produced by the PIT process and having droplet sizes of 50 to 400 nm.

It has surprisingly been found that o/w emulsions produced by the PIT process satisfy the requirements stated above in excellent fashion, allowing for the use of enzymes that do not have sufficient stabililty in previously-known two-phase and other emulsion systems. The PIT system according to the invention has the requisite properties.

### DETAILED DESCRIPTION OF THE INVENTION

Emulsions are disperse preparations of at least two liquids insoluble in one another, of which one liquid contains water. Emulsifiers or emulsifier systems are used to homogenize immiscible o/w phases by emulsification. In the absence of stabilizing emulsifiers, the phases would separate again based on their different polarities. The amphiphilic emulsifiers sit at the interfaces between the fine droplets and the coherent phase and prevent them from coalescing by steric or electrostatic shielding. Emulsifiers are compounds which join hydrophilic and lipophilic structural units to one another in their molecular structure. The choice and extent of the particular structural units in the emulsifier molecule or emulsifier system affected are often characterized by the hydrophilic/lipophilic balance [HLB (number) value]. As a general rule, emulsifiers or emulsifier systems with comparatively strongly hydrophilic components lead to high HLB values and, in their practical application, generally lead to water-based o/w emulsions with a disperse oil phase. Emulsifiers or emulsifier systems with comparatively strongly lipophilic components lead to comparatively lower HLB values and hence to w/o invert emulsions with a continuous oil phase and a disperse water phase.

It is known that oil-in-water (o/w) emulsions prepared and stabilized with nonionic emulsifiers can undergo generally reversible phase inversion on heating, i.e. the emulsion type changes from o/w to w/o within a certain temperature range. Since the oil becomes the outer continuous phase, the conductivity of the emulsion falls to zero. The mean value of the temperatures between maximal and zero conductivity of the emulsion on heating is called the phase inversion temperature (PIT) and the emulsions thus produced are called PIT emulsions.

It is also known that the position of the PIT depends on a number of factors, for example the nature and phase volume of the oil component, the hydrophilicity and the structure of the emulsifiers and the composition of the emulsifier system.

The droplet size of the PIT emulsion is critically determined by the production process. In general, the water and oil phases are mixed with the emulsifiers and then heated to a temperature above the PIT, the conductivity having to fall to zero. The emulsion is then cooled to the starting temperature (generally room temperature, ca. 20° C.). The emulsions used in accordance with the invention are only formed by the temperature of the emulsions exceeding and then falling below the PIT.

It is known that only those PIT emulsions which form a microemulsion phase with low interfacial tension between oil and water or a lamellar liquid-crystalline phase during phase inversion are characterized by particularly small droplets. The crucial step is always the re-inversion on cooling.

The emulsions according to the invention are distinguished in particular by their droplet fineness. The droplet size is between 50 and 400 nm, preferably in the range of from 70 to 300 nm, more preferably in the range of from 80 to 250 nm and most preferably in the range of from 90 to 160 nm. The droplet sizes are assumed to follow a Gauss distribution. They are measured, for example, by light scattering or absorption.

These fine-droplet emulsions retain their homogeneity through Brownian molecular movement (a thermal random movement of particles below 5 μm in size). Brownian motion is the driving force of diffusion and prevents both sedimentation and creaming up (flotation). A major advantage is that the need for energy-intensive stirring may be reduced. It leads to improved diffusion of substrate and enzyme and to reduced energy costs.

The substrate concentrations can be varied without the droplet size having to be reduced. A high substrate concentration can be achieved without any of the droplets coalescing. The low surface tension increases the transfer rate of the molecules at the oil/water interface. The high reproducibility and stability of the PIT emulsions enable biochemical studies to be carried out on enzymes and their reactivity and already known reaction conditions and activities for enzymes to be further optimized.

The emulsions according to the invention are distinguished by the fact that they show adequate stability during the reaction phase in the use according to the invention. This implies that disintegration of the emulsion produced by the PIT process after the desired reaction is not a disadvantage and, in a preferred embodiment, is desired. This has the advantage that the products are easier to work up.

Besides water, the PIT emulsions also contain an oil phase which contains compounds from the group of mineral oils and fatty acid alkyl esters (a) or native vegetable oils and oleochemical derivatives thereof (b). The groups “(a)” and “(b)” are hydrophobic compounds insoluble or only very sparingly soluble in water which may preferably represent the starting materials, i.e. substrates, for the products to be obtained by biocatalytic catalysis, but which may also be used as auxiliaries. The compounds in question are essentially fatty acid esters, fatty alcohol ethers, fatty alcohol esters and fatty acid polyol esters.

Suitable group “(a)” esters are derived in particular from saturated or unsaturated, linear or branched fatty acids containing a total of 7 to 23 carbon atoms. Accordingly, they are compounds corresponding to formula (I):

R¹—COO—R² (I)

in which R¹ is a C₆₋₂₂ alkyl group and R² is a C₁₋₄ alkyl group, methyl and ethyl groups being particularly preferred. The use of methyl esters is the most advantageous. The methyl esters of formula (I) may be obtained in the usual way, for example by transesterification of triglycerides with methanol and subsequent distillation. Suitable fatty acids are caproic, heptanoic, caprylic, pelargonic, capric, undecanoic, lauric, tridecanoic, myristic, pentadecanoic, palmitic, heptadecanoic, stearic, nonadecanoic, arachic and behenic acid. Unsaturated representatives are, for example, lauroleic, myristoleic, palmitoleic, petroselaldic, oleic, elaidic, ricinoleic, linoleic, conjugated linoleic acid (CLA), more particularly cis9,trans11-CLA or trans10, cis12-CLA, linolaidic, linolenic, conjugated linoleic acid, gadoleic, arachidonic and erucic acid. Individual methyl or ethyl esters, as well as mixtures of methyl and/or ethyl esters of these acids are also suitable. It is particularly preferred to use PIT emulsions which contain methyl and/or ethyl esters from the group consisting of methyl oleate, methyl palmitate, methyl stearate, methyl pelargonate, ethyl oleate, ethyl palmitate, ethyl stearate and/or ethyl pelargonate. However, methyl and/or ethyl esters based on the natural fatty acid mixtures obtained, for example, from linseed oil, coconut oil, palm oil, palm kernel oil, olive oil, castor oil, rapeseed oil, soybean oil or sunflower oil (in the case of rapeseed and sunflower oil, new and old plants) may also be used.

Suitable group “(b)” compounds are native oils of vegetable origin and oleochemical derivatives thereof. These are essentially mineral oils, fatty acid esters, fatty acid ethers, fatty alcohol ethers, fatty alcohol esters, fatty acid polyol esters, such as—preferably—triglycerides and triglyceride mixtures, the glycerol being completely esterified with relatively long-chain fatty acids. Particularly suitable vegetable oils are selected from the group consisting of peanut, coconut and/or sunflower oil.

Important constituents of the PIT emulsions used in accordance with the invention are the emulsifiers or emulsifier systems used. Nonionic emulsifiers, more particularly ethoxylated fatty alcohols and fatty acids, are preferably used as emulsifiers. To form PIT emulsions, it is of advantage to use a two-component emulsifier system containing a hydrophilic emulsifier (A) and a hydrophobic co-emulsifier (B). Suitable hydrophilic nonionic emulsifiers (A) are substances which have an HLB value of about 8 to 18. The HLB value (hydrophilic/lipophilic balance) is a value which may be calculated in accordance with the following equation:

HLB=(100−L)/5

where L is the percentage by weight of lipophilic groups, i.e. the fatty alkyl or fatty acyl groups in percent in the ethylene oxide addition products.

Fatty alcohol ethoxylates in the context of the teaching according to the invention correspond to formula (II):

R³—O—(CH₂CH₂O)ₙ-H (II)

in which R³ is a linear or branched, saturated or unsaturated alkyl group containing 6 to 24 carbon atoms and n is a number of 1 to 50. Compounds of formula (II) where n is a number of 1 to 35, are preferred 1 to 15 being particularly preferred. Other particularly preferred compounds of formula (II) are those where R³ is an alkyl group containing 16 to 22 carbon atoms.

The compounds of formula (II) are obtained in known manner by reaction of fatty alcohols under pressure with ethylene oxide, optionally in the presence of acidic or basic catalysts. Typical examples are caproic alcohol, caprylic alcohol, 2-ethyl hexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol, as well as the technical mixtures thereof obtained, for example, in the high-pressure hydrogenation of technical methyl esters based on fats and oils or aldehydes from Roelen's oxosynthesis and as monomer fraction in the dimerization of unsaturated fatty alcohols. Technical fatty alcohols containing 12 to 18 carbon atoms, such as for example coconut oil, palm oil, palm kernel oil or tallow fatty alcohol, are preferred.

Fatty acid ethoxylates which may also be used as emulsifier component (A) preferably correspond to formula (III):

R⁴CO₂(CH₂CH₂O)ₘH (III)

where R⁴ is a linear or branched alkyl group containing 12 to 22 carbon atoms and m is a number of 5 to 50 and preferably 15 to 35. Typical examples are products of the addition of 10 to 30 mol ethylene oxide onto lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachic acid, gadoleic acid, behenic acid and erucic acid and the technical mixtures thereof obtained, for example, in the pressure hydrolysis of natural fats and oils or in the reduction of aldehydes from Roelen's oxosynthesis. Products of the addition of 10 to 30 mol ethylene oxide onto C₁₆₋₁₈ fatty acids are preferably used.

Partial glycerides which may be used as emulsifier component (B) preferably correspond to formula (IV):

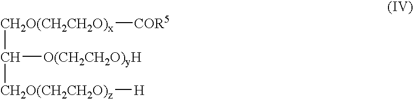

where COR⁵ is a linear or branched acyl group containing 12 to 22 carbon atoms and x+y+z together stand for 0 or for numbers 1 to 50 and preferably 15 to 35. Typical examples of partial glycerides suitable for the purposes of the invention are lauric acid monoglyceride, coconut fatty acid monoglyceride, palmitic acid monoglyceride, stearic acid monoglyceride, isostearic acid monoglyceride, oleic acid monoglyceride, conjugated linoleic acid monoglycerides and tallow fatty acid monoglyceride and addition products thereof with 5 to 50 and preferably 20 to 30 mol of ethylene oxide. Monoglycerides or technical mono/diglyceride mixtures predominantly containing monoglycerides (IV), where COR⁵ is a linear acyl group containing 16 to 18 carbon atoms, are preferably used.

Emulsifier mixtures containing components (A) and (B) in a ratio by weight of 10:90 to 90.10, preferably 25:75 to 75:25 and more particularly 40:60 to 60:40 are normally used.

Other suitable emulsifiers are, for example, nonionic surfactants from one of the following groups:
products of the addition of 2 to 30 mol ethylene oxide and/or 0 to 5 mol propylene oxide onto linear fatty alcohols containing 8 to 22 carbon atoms;
glycerol monoesters and diesters and sorbitan monoesters and diesters of saturated and unsaturated fatty acids containing 6 to 22 carbon atoms and ethylene oxide adducts thereof;
alkyl mono- and oligoglycosides containing 8 to 22 carbon atoms in the alkyl group and ethoxylated analogs thereof;
products of the addition of 15 to 60 mol ethylene oxide onto castor oil and/or hydrogenated castor oil;
polyol esters and, in particular, polyglycerol esters such as, for example, polyglycerol polyricinoleate or polyglycerol poly-12-hydroxy-stearate. Mixtures of compounds from several of these classes are also suitable;
products of the addition of 2 to 15 mol ethylene oxide onto castor oil and/or onto hydrogenated castor oil;
partial esters based on linear or branched, unsaturated or saturated C₆₋₂₂ fatty acids, ricinoleic acid and 12-hydroxystearic acid and glycerol, polyglycerol, pentaerythritol, dipentaerythritol, sugar alcohols (for example sorbitol) and polyglucosides (for example cellulose);
wool wax alcohols; or
polyalkylene glycols.

The addition products of ethylene oxide and/or propylene oxide onto glycerol mono- and diesters and sorbitan mono- and diesters of fatty acids or onto castor oil or hydrogenated castor oil are known commercially-available products. They are homolog mixtures of which the average degree of alkoxylation corresponds to the ratio between the quantities of ethylene oxide and/or propylene oxide and substrate with which the addition reaction is carried out.

To select suitable emulsifier systems, it can be useful to calculate the PIT of the particular system. However, this also applies in particular to potential optimizations in the choice of the emulsifiers or emulsifier systems and their adaptation to the choice and mixing of aqueous phase on the one hand and the type of oil phase on the other hand as predetermined by other considerations as to technical procedure. Corresponding expert knowledge has been developed in totally different fields, particularly in the production of cosmetics. Particular reference is made in this connection to the article by T H. Förster, W. von Rybinski, H. Tesmann and A. Wadle “Calculation of Optimum Emulsifier Mixtures for Phase Inversion Emulsification” in International Journal of Cosmetic Science 16, 84-92 (1994), which contains a detailed account of how the phase inversion temperature (PIT) range of a given three-component system of an oil phase, a water phase and an emulsifier may be calculated by the CAPICO method (calculation of phase inversion in concentrates) on the basis of the EACN value (equivalent alkane carbon number) characteristic of the oil phase. More particularly, this article by Förster et al. cites important literature on the subjects under discussion here which should be viewed in conjunction with the disclosure of this article by Förster et al. With the aid of numerous examples, it is shown how the choice and optimization of the emulsifiers/emulsifier systems are accessible to the adjustment of optimal predetermined values for the phase inversion temperature range by the CAPICO method in conjunction with the EACN value.

The PIT emulsions used in accordance with the present invention preferably contain 20 to 90% by weight of water, more preferably 30 to 80% by weight and most preferably 30 to 60% by weight of water. The balance to 100% by weight is made up of oil phase and emulsifiers and optionally other auxiliaries and additives. The oil phase itself is present in quantities of preferably 10 to 80% by weight and more particularly 40 to 70% by weight. In a preferred embodiment, the oil phase exclusively contains hydrophobic components (a) (esters) or (b) (native vegetable oils and oleochemical derivatives thereof) or mixtures of these components. The emulsifiers or emulsifier systems are present in quantities of preferably 1 to 25% by weight, more preferably 5 to 20% by weight and most preferably 5 to 15% by weight. The emulsions used in accordance with the invention preferably have phase inversion temperatures of 20 to 95° C. and more particularly in the range from 30 to 95° C.

The emulsions used in accordance with the invention are developed according to the properties of the educts through the correct choice and composition of oil component and emulsifiers.

Emulsions containing fatty acid alkyl esters or fatty alcohol ethers and an emulsifier mixture based on ethoxylated hydroxyfatty acid triglycerides are preferably used.

In the context of the invention, the biocatalysts to be used in accordance with the invention are capable of catalytically reacting at interfaces, and are understood to be enzymes or whole cells or cell parts, preferably isolated enzymes. Enzymes from the group of lyases and/or oxidoreductases are preferred and may be used either individually or in combination with several enzymes.

Under the IUBMB classification, lyases are representatives of the fourth main group of enzymes. There are four important sub-classes (C—C, C—O, C—N, and C—S lyases, EC 4.1 to 4.4). Lyases non-hydrolytically split off certain groups from, or attach them to, their substrate, leaving a double bond behind or adding groups onto double bonds.

The oxidoreductases represent the first of the six main groups of enzymes; they catalyze redox reactions. The sub-group is generally determined by the nature of the electron donor and is in turn divided into subgroups according to the nature of electron acceptor. The systematic names are formed according to the pattern: donor:acceptor-oxidoreductase.

Lyases selected from the group formed by hydroxynitrilases, nitrilases, nitrile hydratases, oxynitrilases, carboxylases and aldolases are preferred for the purposes of the invention. The oxidoreductases are preferably selected from the group consisting of dehydrogenase, hydroxylase, laccase, lipoxygenase, reductase, oxidase, peroxidase and oxygenase. Hydroxynitrile lyases are particularly preferred enzymes as the biocatalysts.

Typical examples of suitable enzymes, which are not meant to be limiting in any way, are lyases and/or oxidoreductases from organisms selected from the group consisting of Alcaligenes, Aspergillus, Aeromonas aerophila, Bacillus, Candida, Chromobacterium viscosum, Fusarium solani, Geotrichum candidum, Hevea, Issatchenkia orientalis (Candida krusei), Kluyveromyces marxianus (C. kefyr C. pseudotropicalis), Linum, Manihot, Mucor javanicus, Nocarida, Penicilium camemberti Penicilium roqueforti, Pichia, Pseudomonas, Prunus, Rhizomucor, Rhizopus, Sorghum and Thermomyces and mixtures thereof. Lyases and/or oxidoreductases from the organisms Alcaligenes, Candida, Chromobacterium, Nocardia, Rhizomucor, Prunus, Linum, Sorghum, Hevea, Manihot, Pseudomonas, Rhizopus and Thermomyces are preferred. Enzymes from Prunus amygdalus, Prunus serotina, Prunus domestics, Prunus avium, Prunus persica, Malus pumila, Linum usitatissimum, Sorghum bicolor, Hevea brasiliensis and Manihot esculenta are particularly preferred.

The enantioselective enzymes are preferred. (S)-selective hydronitrilase from leaves of Hevea brasiliensis and the (R)-selective hydroxynitrilase from Prunus amygdalus are particularly preferred.

In a preferred embodiment, the lyases are of vegetable origin. In this case, they may be isolated from all constituents of the respective plants, preferably both from the leaves, the stem or stalk and from the fruit.

The oxidoreductases are preferably of microbial origin.

The enzymes to be used in accordance with the invention may be used in various forms. In principle, any supply forms of enzymes known to the expert may be used. In the context of the invention, the definition of “enzyme” also encompasses protein and enzyme protein. Both the enzyme protein and the whole protein, which contains the function of the protein according to the invention in a part of the protein sequence, may be used in accordance with the invention. The enzymes are preferably used in pure form or as a technical enzyme preparation either immobilized on a carrier material and/or in solution, more particularly in aqueous solution, and re-used in so-called repeated batches. Crystallized enzymes, so-called CLECs, obtainable for example from the Altus Company, are also preferred. The percentage of active enzyme in the particular technical enzyme preparations varies from manufacturer to manufacturer. However, the average is between 1 and 10% active enzyme.

In another embodiment of the invention, the enzymes to be used in accordance with the invention are used with an activity of 20 to 5,000 U/ml aqueous phase, expressed as pure enzyme or enzyme preparation. More particularly, the activities to be used are in the range from 30 to 3,000 U/mi aqueous phase.

According to the invention, the biocatalytic reactions are preferably C—C linkages, C—N linkage, C—O linkage or C—S linkage. According to the invention, preferred reactions are enantioselective reactions which lead to chiral compounds obtained through the use of enantioselective enzymes, with an enantiomeric purity of 98 to 99 ee. According to the invention, the production of chiral compounds is preferred.

According to the invention, fine chemicals as intermediate products for cosmetic and/or pharmaceutical products and/or as intermediate products for agricultural applications are produced by the biocatalytic reactions using the PIT emulsions according to the invention. More particularly, the fine chemicals are cyanhydrins, more especially enantiomer-pure cyanhydrins. The production of enantiomer-pure cyanhydrins both in the (S) and in the (R) configuration opens up numerous possibilities for stereoselective follow-on reactions, such as for example hydrolysis to chiral hydroxy acids.

The o/w emulsions according to the invention containing at least water, emulsifiers and an oil phase and produced by the PIT process are eminently suitable for use as a reaction medium for biocatalytic reactions. Accordingly, the present invention also relates to a process for biocatalytic C—C linkages, C—N linkage, C—O linkage or C—S linkage, in which o/w emulsions produced by the PIT process are used as the reaction medium. The emulsions used for the process according to the invention correspond in their constituents, conditions and more detailed embodiments of the emulsions which have already been described in detail for the use of these o/w emulsions. In this process, use may be made of the fact that the water-insoluble substances become soluble in the oil phase of the PIT emulsion and are thus accessible to the enzyme-catalyzed reaction.

According to the invention, the PIT emulsions containing the substrate are added to the reaction vessel containing the immobilized or non-immobilized biocatalysts and optionally other auxiliaries and additives. The details of this process, more particularly the quantity of biocatalyst and the added emulsion, are determined by the nature of the biocatalyst and the PIT emulsion selected and may be adapted by the practitioner to suit the particular circumstances. By heating the system, phase separation may be achieved and the product in the oil phase may readily be separated from the aqueous phase. By using fixed-bed reactors containing the enzyme, the enzyme can be removed and re-used.

The fineness of the oil droplets leads to a large surface between the oil phase and the water phase and thus provides for rapid contact and an increased reaction rate between the biocatalysts and the oil phase containing the substrates.

In one particular embodiment of this process, the enzymes used are the enzymes already listed for the use of the o/w emulsion according to the invention.

The reaction conditions according to the invention for the biocatalytic reaction are determined by the optimal reaction range of the enzymes selected and the emulsions used. More particularly, the reaction temperature, inter alias, is between 4 and 50° C. and preferably between 15 and 40° C. and, more particularly, is 20° C.

In the event of unwanted secondary reactions that are not caused by the biocatalyst, the temperature can be lowered to between 0° C. and 20° C. to reduce the secondary reactions. In such cases, a temperature of 3° C. to 15° C. is preferred.

### EXAMPLES

### Production Example H1

### Enzymes and Chemicals

R-oxynitrilase (EC 4.1.2.10, R-mandelonitrile lyase, R-acetone cyanhydrin lyase) from Prunus amygdalus (38 U/ml solution, FLUKA);
R-(+)-mandelonitrile (>99%, Aldrich); benzaldehyde (>99%, redistilled, Aldrich); KCN (analytical grade, FLUKA), sodium phosphate (Prolabo); Eumulgin HRE 40 (from Cognis); methyl oleate (from Cognis); Cetiol® OE (INCI: Dicaprylyl Ether, from Cognis); ultrapure water (milipore milliQ+); ethanol (Carlo Erba).

### PIT Emulsions:

Two PIT emulsions were prepared. One contained benzaldehyde dissolved in methyloleate, the other contained benzaldehyde dissolved in Cetiol® OE. The final composition was: 0.5 g methyl oleate or Cetiol OE/0.5 g Eumulgin® HRE 40/0.4 ml benzaldehyde/1.4 ml water. In addition, the PIT emulsion was diluted by addition of 1 ml water. Both emulsions remained stable for weeks at 4° C. Fresh emulsions were prepared for each experiment in order to avoid the degradation of benzaldehyde.

### Reaction:

The reaction medium was formed by: 1.2 ml phosphate buffer (pH 5.7, 50 mM); 0.4 ml cyanide solution, (5 g KCN in 10 ml water); 0.2 ml PIT emulsion; 0.2 ml (7.6 U) enzymes or 0.2 ml buffer. The reactions were conducted in closed (Teflon layer) 4 ml Wheaton™ bottles without stirring except for the random sample.

After various reaction times, 10 μl samples were taken from each reaction medium and added to 1.5 ml ethanol in microtubes. The tubes were centrifuged for 2 minutes at 10,000×G in order to separate the protein deposits from the medium containing enzymes. The absorption capacity of the supernatant at 283 nm was measured in pairs in quartz cells in a Beckman DU 530 spectrophotometer.

### Results and Discussion:

A spectrophotometric method based on a specific absorption maximum of benzaldehyde at 283 nm was used to determine the reaction kinetics. The absorption spectrum in ethanol of benzaldehyde and the reaction product mandelonitrile (20× more concentrated) was determined.

The extinction coefficient at 283 nm of benzaldehyde in ethanol was determined at 1130 m-¹ cm-¹.

Two independent series of experiments were carried out. Each series compared reactions with and without enzymes for PIT systems containing Eumulgin® HRE 40, benzaldehyde and either methyloleate or Cetiol® OE. Reactions of the first series were carried out for 2 hours at 20° C. and then at 4° C. The second series was incubated at 4° C. for the entire duration. The very low reaction temperature (4° C.) was selected in order to limit the uncatalyzed reactions. Without cyanides, no reaction was observed. It is pointed out that a yellow-orange color develops in the tubes containing cyanides (with or without enzymes), even when citrate buffer was exchanged for phosphate buffer. This color presumably corresponds to the absorption peak with a maximum of 385. The coloration was weaker with phosphate buffer than with citrate buffer. Experiments showed that these colors also developed without PIT emulsions and also without enzymes which could indicate a chemical reaction with the impurities of the buffer compositions.

The kinetics were determined for experiments at 20° C. and then 4° C. and for experiments at 4° C. The difference in absorption capacity at 283 nm between reaction medium with and reaction medium without enzymes was determined. This difference coincides with the absorption maximum of benzaldehyde. Enzyme-catalyzed bioconversions of benzaldehyde were observed in this way with both benzaldehyde/methyloleates and benzaldehyde/Cetiol® OE PIT emulsions. The reaction rate of the benzaldehyde conversion was 0.30 μmol/ml/h in the presence of benzaldehydes/Cetiol® OE systems at 4° C. and 0.14 μmol/ml/h with benzaldehyde/methyloleates at 4° C.

| | | Conversion rate in μmol/ml*h |
|---|---|---|
| | | Enzyme contribution (difference in |
| | | benzaldehyde consumption with and |
| System | Temperature | without enzyme) |
| PIT: Benzaldehyde/ | 4° C. | 0.14 |
| Methyloleate/ | | |
| Eumulgin and water | | |
| PIT: Benzaldehyde/ | 4° C. | 0.3 |
| Cetiol ®OE/ | | |
| Eumulgin ® and | | |
| water | | |

The reactions take place at least 20 times faster at 20° C. The data show that an enzyme conversion is possible in PIT systems containing a lyase.

### Further Results:

The same reaction was carried out with lyase from Hevea brasiliensis at low temperatures and without stirring. With this lyase, too, the reaction of benzaldehyde with cyanide could be successfully carried out in a PIT emulsion. Benzaldehyde was dissolved either in methyloleate or in Cetiol® OE. The reaction in the presence of benzaldehyde/Cetiol® OE was twice as fast as the reaction in methyloleate.

1-23. (canceled)

24: An oil-in-water emulsion reaction medium for biocatalytic reactions comprising water, one or more emulsifiers, and an oil phase, characterized in that the emulsion is produced by the PIT process and has a droplet size of 50 to 400 nm.

25: The oil-in-water emulsion reaction medium according to claim 24, characterized in that the oil phase comprises one or more compounds selected from the group consisting of mineral oils, fatty acid alkyl esters, fatty alcohol ethers, fatty alcohol esters and fatty acid polyol esters.

26: The oil-in-water emulsion reaction medium according to claim 24, characterized in that the emulsion produced by the PIT process is stable during the reaction phase.

27: The oil-in-water emulsion reaction medium according to claim 24, characterized in that emulsions containing one or more fatty acid alkyl esters corresponding to formula (I):
R¹—COO—R² (I)
in which R¹ is a C₆₋₂₂ alkyl group and R² is a C₁₋₄ alkyl group, are used.

28: The oil-in-water emulsion reaction medium according to claim 24, characterized in that emulsions containing the oil phase in quantities of 10 to 80% by weight are used,

29: The oil-in-water emulsion reaction medium according to claim 24, characterized in that emulsions containing water in quantities of 20 to 90% by weight, are used.

30: The oil-in-water emulsion reaction medium according to claim 24, characterized in that emulsions containing one or more fatty acid alkyl esters or one or more fatty alcohol ethers and an emulsifier mixture based on ethoxylated hydroxyfatty acid triglycerides as the one or more emulsifiers, are used.

31: The oil-in-water emulsion reaction medium according to claim 24, characterized in that emulsions which comprise an emulsifier system containing one or more hydrophilic emulsifiers with (an) HLB value(s) of 8 to 18 in combination with one or more hydrophobic co-emulsifiers, is used.

32: The oil-in-water emulsion reaction medium according to claim 31, characterized in that the emulsifier system having quantity ratios between hydrophilic emulsifiers and hydrophobic co-emulsifiers of 10:90 to 90:10 is used.

33: The oil-in-water emulsion reaction medium according to claim 24, characterized in that emulsions containing the one or more emulsifiers in quantities in the aggregate of 1 to 25% by weight, are used.

34: The oil-in-water emulsion reaction medium according to claim 24, also comprising one or more enzymes selected from the group comprising one or more lyases, one or more oxidoreductases, and mixtures of one or more lyases and one or more oxidoreductases.

35: The oil-in-water emulsion reaction medium according to claim 34, characterized in that the one or more lyases is/are selected from the group consisting of hydroxynitrilases, nitrilases, nitrile hydratases, and oxynitrilases and the one or more oxidoreductases is/are selected from the group consisting of dehydrogenase, hydroxylase, laccase, lipoxygenase, reductase, oxidase, peroxidase and oxygenase.

36: The oil-in-water emulsion reaction medium according to claim 34, characterized in that the one or more lyases and/or the one or more oxidoreductases is/are obtainable from organisms selected from the group consisting of Alcaligenes, Aspergillus, Aeromonas aerophila, Bacillus, Candida, Chromobacterium viscosum, Fusarium solani, Geotrichum candidum, Hevea, Issatchenkia orientalis (Candida krusei), Kluyveromyces marxianus (C. kefyr, C. pseudotropicalis), Linum, Manihot, Mucor javanicus, Nocarida, Penicilium camemberti, Penicilium roqueforti, Pichia, Pseudomonas, Prunus, Rhizomucor, Rhizopus, Sorghum and Thermomyces and mixtures of two or more thereof.

37: The oil-in-water emulsion reaction medium according to claim 34, characterized in that the lyase(s) is/are of vegetable origin.

38: The oil-in-water emulsion reaction medium according to claim 34, characterized in that the enzyme(s) used has/have an activity of 20 to 5,000 U/ml aqueous phase expressed as pure enzyme or as enzyme preparation.

39: The oil-in-water emulsion reaction medium according to claim 24, characterized in that the biocatalytic reactions are C—C linkage, C—N linkage, C—O linkage or C—S linkage reactions.

40: The oil-in-water emulsion reaction medium according to claim 24, characterized in that chiral compounds are produced by enantioselective enzymes in the biocatalytic reaction.

41: The oil-in-water emulsion reaction medium according to claim 24, characterized in that fine chemicals as intermediate products for cosmetic and/or pharmaceutical products and/or as intermediate products for agricultural applications are produced in the biocatalytic reaction.

42: The oil-in-water emulsion reaction medium according to claim 41, characterized in that the fine chemicals are cyanhydrins.

43: A process for biocatalytic C—C linkage, C—N linkage, C—O linkage or C—S linkage reactions, characterized in that oil-in-water emulsions are used as the reaction medium according to claim 1.

44: The process according to claim 43, characterized in that fine chemicals as intermediate products for cosmetic and/or pharmaceutical products and/or as intermediate products for agricultural applications are produced in the biocatalytic reaction.
